(19) United States
(12) Patent Application Publication
(10) Pub. No.: US 2011/0311470 A1
(43) Pub. Date: Dec. 22, 2011

(54) COMPOSITIONS FOR A POWDER HAVING AN AQUEOUS PHASE

(75) Inventors: JANINE CHERETTE, MIDDLESEX, NJ (US); DHAVAL PATEL, EDISON, NJ (US); ASHINI AMIN, MONROE, NJ (US)

(73) Assignee: L'ORÉAL S.A., PARIS (FR)

(21) Appl. No.: 13/152,694

(22) Filed: Jun. 3, 2011

Related U.S. Application Data
(60) Provisional application No. 61/356,245, filed on Jun. 18, 2010.

(51) Int. Cl. A61K 8/55; A61Q 1/12

(52) U.S. Cl. 424/69

(57) ABSTRACT

In an embodiment of the present invention, there is provided a personal care composition having: a) at least one hydrophilic binder chosen from a sodium starch glycolate cross-linked with a phosphate ester group; b) at least one hydrophobic powder filler; and c) water, wherein the composition is in loose powder form. In another embodiment of the present invention, there is provided a method of making a personal care composition involving: a) forming a powder phase, comprising: i) at least one hydrophilic binder chosen from a sodium starch glycolate cross-linked with a phosphate ester group; and ii) at least one hydrophobic powder filler; b) providing an aqueous phase, comprising water; and c) adding the aqueous phase to the powder phase, wherein the personal care composition is in loose powder form.

### CROSS-REFERENCE TO RELATED APPLICATION

This application claims the benefit of the filing date of U.S. Provisional Application No. 61/356,245, filed Jun. 18, 2010, the contents of which are incorporated by reference.

### FIELD OF THE INVENTION

The present invention relates to personal care products, particularly those in loose powder form. Specifically, the present invention relates to a powder composition having an aqueous phase that provides a cooling, fresh feeling for the user.

### BACKGROUND OF THE INVENTION

Powders have been utilized in personal care products for numerous applications. In particular, powders have been utilized for deodorants, cosmetics, dry shampoos and the like. Pressed and loose powders are well known in the industry and have been known to provide a fresh, light feeling to the skin. However, powders have some drawbacks.

Traditionally, powders have been anhydrous and have been known to cause a drying feeling upon application to the skin. In an effort to reduce or eliminate the drying feeling, the addition of water has been contemplated. However, the addition of water has led to problems of stability and the water has a tendency to evaporate easily. Further, powders that incorporate water have shown a tendency to clump or form into a gel or cream, losing their powder form. Thus, it has generally been difficult to incorporate water into a powder.

Accordingly, it is desirable to provide a product in the form of a powder that has a fresh, light feeling without any attendant dryness. It is also desirable to provide a powder composition that incorporates an aqueous phase without the powder losing its form.

### SUMMARY OF THE INVENTION

In an embodiment of the present invention, there is provided a personal care composition having: a) at least one hydrophilic binder chosen from a sodium starch glycolate cross-linked with a phosphate ester group; b) at least one hydrophobic powder filler; and c) water, wherein the composition is in loose powder form.

In another embodiment of the present invention, there is provided a method of making a personal care composition involving: a) forming a powder phase, having: i) at least one hydrophilic binder chosen from a sodium starch glycolate cross-linked with a phosphate ester group; and ii) at least one hydrophobic powder filler; b) providing an aqueous phase, comprising water; and c) adding the aqueous phase to the powder phase, wherein the personal care composition is in loose powder form.

In yet another embodiment of the present invention, there is provided a method of treating a keratinous substrate involving applying onto the substrate a composition having: a) at least one hydrophilic binder chosen from a sodium starch glycolate cross-linked with a phosphate ester group; b) at least one hydrophobic powder filler; and c) water, wherein the composition is in loose powder form.

It has been surprisingly found that by employing a specific type of binder, a loose powder can incorporate large amounts of water while maintaining its powder form. The resultant composition possesses a light, fresh feeling and imparts a cooling sensation to the skin upon application. Further, it has been surprisingly found that the loose powder composition maintains its powder form upon application to a keratinous substrate.

### DETAILED DESCRIPTION OF THE INVENTION

Other than in the operating examples, or where otherwise indicated, all numbers expressing quantities of ingredients and/or reaction conditions are to be understood as being modified in all instances by the term “about,” meaning within 10% to 15% of the indicated number.

The invention is directed to a powder for personal care compositions having an aqueous phase incorporated therein. Such compositions have a fresh, cooling feeling upon application to a keratinous substrate, such as the skin. The result is a natural, healthy looking skin appearance. The powder of the present invention is suitable for application to any area of the skin, such as the face, lips, eyes, scalp, hair, eyelashes, or any other body area.

Generally, the compositions of the present invention have a powder phase that includes fillers that are either hydrophobic in nature or are hydrophobically modified. The powder phase also includes a hydrophilic binder. An aqueous phase is then added to the powder phase, allowing the hydrophilic binder to fully absorb and retain the water, causing the binder to swell with the water, while maintaining the loose powder form.

Powder Phase

Binder

The compositions of the present invention utilize a hydrophilic binder that is capable of absorbing water without clumping, or forming a cream upon application onto a keratinous substrate. The binder used herein is a sodium starch glycolate (“SSG”), produced by cross-linking and carboxymethylation of a starch. The starch source, degree of cross-linking and the degree of substitution allow for such water absorption without the formation of a gel or cream. The loose powder form of the compositions is maintained even after application onto a keratinous substrate.

Although a wide variety of starches may be employed, potato starch is preferred. Potato starch showed the highest water uptake and provided the fastest disintegration. The starch is then modified through cross-linking and substitution to optimize the level of water retention. The modifications include cross-linking of the polymer backbone and the introduction of carboxymethyl sodium groups.

In particular, the starch is cross-linked using a starch esterifying agent, such as sodium trimetaphosphate or phosphorus oxychloride in alkaline suspension. These starch esterifying agents form cross-linked distarch phosphate. Thus, the starch esterifying agents introduce strong phosphate ester links into the starch. The phosphate ester links are short, providing for a tight, dense network.

Further, it is preferred to keep the degree of cross-linking low, for example, one phosphate ester group per 500 anhydroglucose units. The low degree of cross-linking reduces the water soluble fraction of the starch and the viscosity of the dispersion in the water.

The cross-linked starch is then substituted using chloroacetic acid or sodium monochloroacetate in an alkaline alcoholic suspension. The degree of substitution is in the range of about 0.23 to about 0.32. Thus, about 1 in every 4 glucose units are substituted with carboxymethyl sodium groups. Without intending to be bound by theory, it is believed that these hydrophilic carboxymethyl groups disrupt the hydrogen bonding that holds the starch together, allowing the water to penetrate the starch.

It is also believed that this allows the starch to dissolve in cold or room temperature water. Incorporation of traditional starch into personal care compositions generally requires heat because-starch is not normally soluble in cold or room temperature water. Formation of the composition in cold water or room temperature water is desirable from an energy efficiency and sustainability standpoint. Further, cold or room temperature water processing is desirable because it eliminates complexity during manufacturing.

After substitution, the mixture is then neutralized and the SSG is isolated and dried and has a loose powder form. The amount of water uptake by SSG is about 20 g/g, that is, it can absorb about 20 times its weight in water. The molecular weight of the SSG of the present invention is generally the same as the molecular weight of the original starch, ranging from about 20,000 Da to 200,000,000 Da, preferably about 30,000,000 Da.

Compositions formed with other sodium starch glycolates known in the art show a decrease in viscosity over time and turn into a cream upon application onto a keratinous substrate. These other SSGs use carboxylate groups to cross-link the starch molecules. Because the ester links are long, a loose cross-linked network is formed that permits a large amount of water absorption.

Further, it has been found that SSG cross-linked with carboxylate groups shows a decrease in viscosity over time. It is believed that this is due to the carboxylate cross-links being susceptible to hydrolysis. Over time, hydrolysis lowers the viscosity of the composition. Thus, a loose powder form is not maintained over time.

Further, upon application to the skin, the SSG cross-linked with carboxylate groups, forms a cream, because the larger cross-linked network, holding more water, releases the water under shear, as when a user applies the composition onto the skin.

Thus, the SSG cross-linked with carboxylate retains more water, shows a decrease in viscosity over time and forms a cream upon application onto a keratinous substrate.

In contrast, the SSG cross-linked with the phosphate ester links, of the present invention, does not suffer from these drawbacks. The phosphate links allow the SSG to have a dense cross-linked structure that prevents the SSG from absorbing a large amount of water. Further, the phosphate links are not susceptible to hydrolysis. Thus, the SSG cross-linked with the phosphate ester links does not show a decrease in viscosity over time. Lastly, a loose powder form is maintained upon application to a keratinous substrate because a large amount of water is not absorbed.

The SSG cross-linked with phosphate ester links of the present invention is available as PRIMOJEL® from DMV-Fonterra Excipients. The SSG is employed in an amount ranging from about 0.1 to about 20% by weight, preferably from about 0.1 to about 10% by weight, and more preferably from about 0.1 to about 5% by weight, including all ranges and subranges therebetween, all weights based on the total weight of the composition.

Filler

The present invention also includes at least one hydrophobic filler powder. In particular, the filler powder is either hydrophobic in nature or hydrophobically treated. Suitable filler powders include, but are not limited to, mineral silicate, talc, serecite, zinc oxide, titanium dioxide, synthetic polymer powder, and the like, that are known and utilized in the art.

In particular, mica is preferred. Although mica is hydrophilic by nature, the preferred mica is hydrophobically treated. It is known that a suitable hydrophobic treatment of mica includes treatment with lauroyl lysine.

The at lease one hydrophobic powder filler is employed in an amount ranging from about 0.1 to about 99% by weight, preferably from about 0.1 to about 80% by weight, and more preferably from about 1 to about 70% by weight, including all ranges and subranges therebetween, all weights based on the total weight of the composition.

Aqueous Phase

Water

The compositions of the present invention also contain water. The water is present in an amount of from about 5 to about 90% by weight, preferably from about 5 to about 50% by weight, and more preferably from about 5 to about 30% by weight, including all ranges and subranges therebetween, all weights based on the total weight of the composition.

In a preferred embodiment, the aqueous phase and the powder phase are employed in a ratio by weight of about 4:1 to about 1:1, and preferably from about 3:1 to about 1:1 and more preferably from about 2.5:1 to about 1:1.

Auxiliaries

Various types of auxiliary ingredients may be used in the composition of the present invention. Suitable ingredients include, for example colorants such as pigments, inks and lakes; dermatological ingredients such as sunscreen agents, anti-acne agents, anti-aging compounds; insect repelling agents; transdermal pharmaceutical compounds; deodorant and antiperspirant agents; perfumes; dye compounds; texturizing agents, such as polymethyl methacrylates; minerals for luminosity and radiance; emollients; humectants; preservatives; etc. The type and amount of optional ingredient to be employed will depend on the composition's ultimate use, and is to be determined by those of ordinary skill in the art. The incorporation of the optional ingredient into either phase is also determined by those of ordinary skill in the art.

Method of Making

In an embodiment of the present invention, a method of making the compositions of the present invention is provided. It is preferred to form the resultant composition by combining two phases: a powder phase and an aqueous phase.

It is preferred to form a powder phase comprising the binder, filler powder and any other components that are desired. The powder phase can include various components, so long as the resultant powder phase remains in a loose powder form and the components are either hydrophobic in nature or hydrophobically modified. Suitable components for the powder phase can include, but are not limited to, pigments, texturizing agents such as polymethyl methacrylates, preservatives, minerals, emollients such as silicone oil, and the like. Thus, only the binder is hydrophilic in the powder phase.

Next an aqueous phase consisting primarily of water is formed. In this phase, it may be preferred to incorporate preservatives, actives, humectants, and the like, as desired.

The aqueous phase is then added to the powder phase, allowing all the hydrophobic components of the powder phase to repel the water, so that the hydrophilic SSG of the binder can absorb all of the water and swell.

The addition of the aqueous phase to the powder phase is performed in a metal container, preferably a stainless steal container. Glass and certain plastics have an affinity for water, which prevents all of the water from being fully absorbed by the binder. Thus, the formation of the final composition is performed in a metal container, or one that shows no affinity for water.

Likewise, the final composition is packaged in a container that shows no affinity for water because the water may escape the binder over time due to such an affinity. Further, an air-tight container is required to prevent evaporation of the water. Suitable packaging materials include, but are not limited to, polyethylene or polyethylene terepthalate glycol.

The resultant compositions are a loose powder that has water incorporated therein. Upon application to a keratinous substrate, a cooling feeling is imparted by the water. Personal care compositions which may be formulated using the compositions of the present invention can include, but are not limited to, colored cosmetics such as foundation, eyeshadow, blush, deodorants, antiperspirants, dry shampoos, and the like.

The present invention is further described in terms of the following non-limiting example. Unless otherwise indicated, all parts and percentages are on a weight-by-weight percentage basis.

### EXAMPLE

Powder Composition

| Phase | INCI Name | % w/w |
|---|---|---|
| A | Powder Phase | 69.07 |
| B | Aqueous Phase | 30.93 |
| | Total | 100 |

Powder Phase

| | INCI Name | | % w/w |
|---|---|---|---|
| A1 | Filler | 24.33 | 35.23 |
| | Soft | 3.74 | 5.41 |
| | Focus/Texture | | |
| | Ingredient | | |
| | Preservative | 0.35 | 0.51 |
| | Active | 0.10 | 0.14 |
| | Ingredient | | |
| | Mineral Pigment | 12.00 | 17.37 |
| A2 | Colorants | 25.00 | 36.2 |
| A3 | Emollient | 1.50 | 2.17 |
| A4 | Sodium starch | 2.05 | 2.97 |
| | glycolate | | |
| | (PRIMOGEL ®) | | |
| | Total | 69.07 | 100 |

Aqueous Phase

| | INCI Name | | % wt/wt |
|---|---|---|---|
| B | Water | 26.87 | 86.87 |
| | Preservatives | 1.05 | 3.39 |
| | Water Soluble | 0.50 | 1.62 |
| | Active Ingredients | | |
| | Humectant | 2.50 | 8.08 |
| | pH Adjuster | 0.01 | 0.03 |
| | Total | 30.93 | 100 |

Procedure

1. Mixed aqueous phase B until all components were well dissolved and set aside.

2. Mixed phases A1+A2 using an Osterizer blender for 2 minutes on high speed.

3. Added phase A3 and mixed for 2 minutes on low speed.

4. Added phase A4 and mixed for 1 minute on low speed.

5. Added powder phase A to a stainless steel beaker.

6. At a medium steady stream, added the aqueous phase B to the powder phase A under high shear using a marine propeller blade.

7. Mixed until the aqueous phase B was dispersed throughout the powder phase A and retained a powder form.

1) A personal care composition, comprising:
a) at least one hydrophilic binder chosen from a sodium starch glycolate cross-linked with a phosphate ester group;
b) at least one hydrophobic powder filler; and
c) water, wherein the composition is in loose powder form.

2) The composition of claim 1, wherein the composition is in loose powder form prior to application onto a keratinous substrate and remains in loose powder form after application onto the keratinous substrate.

3) The composition of claim 1, wherein the sodium starch glycolate is substituted with a carboxymethyl sodium group.

4) The composition of claim 3, wherein the sodium starch gylcolate has a degree of substitution in the range of about 0.23 to about 0.32.

5) The composition of claim 1, wherein (a) is employed in an amount of from about 0.1 to about 20% by weight, based on the weight of the composition.

6) The composition of claim 1, wherein (b) is a hydrophobically modified mica.

7) The composition of claim 1, wherein (b) is employed in an amount of from about 0.1 to about 99% by weight, based on the weight of the composition.

8) The composition of claim 1, wherein (c) is employed in an amount of from about 5 to about 90% by weight, based on the weight of the composition.

9) A method of making a personal care composition, comprising:
a) forming a powder phase, comprising:
i) at least one hydrophilic binder chosen from a sodium starch glycolate cross-linked with a phosphate ester group; and
ii) at least one hydrophobic powder filler;
b) providing an aqueous phase, comprising at least water; and
c) adding the aqueous phase to the powder phase, wherein the personal care composition is in loose powder form.

10) The method of claim 9, wherein the composition is in loose powder form prior to application onto a keratinous substrate and remains in loose powder form after application onto the keratinous substrate.

11) The composition of claim 9, wherein the sodium starch glycolate is substituted with a carboxymethyl sodium group.

12) The composition of claim 11, wherein the sodium starch gylcolate has a degree of substitution in the range of about 0.23 to about 0.32.

13) The method of claim 9, wherein (a) is employed in an amount of from about 0.1 to about 20% by weight, based on the weight of the composition.

14) The method of claim 9, wherein (b) is a hydrophobically modified mica.

15) The method of claim 9, wherein (b) is employed in an amount of from about 0.1 to about 99% by weight, based on the weight of the composition.

16) The method of claim 9, wherein (c) is employed in an amount of from about 5 to about 90% by weight, based on the weight of the composition.

17) A method of treating a keratinous substrate comprising applying onto the substrate a composition comprising:
a) at least one hydrophilic binder chosen from a sodium starch glycolate cross-linked with a phosphate ester group;
b) at least one hydrophobic powder filler; and
c) water, wherein the composition is in loose powder form.

18) The method of claim 17, wherein treating may be chosen from imparting color, deodorizing, cleaning, applying at least one active ingredient, and protecting from environmental damage.

19) The method of claim 17, wherein the composition is in loose powder form prior to application onto a keratinous substrate and remains in loose powder form after application onto the keratinous substrate.

20) The composition of claim 17, wherein the sodium starch glycolate is substituted with a carboxymethyl sodium group.

21) The composition of claim 20, wherein the sodium starch gylcolate has a degree of substitution in the range of about 0.23 to about 0.32.

22) The method of claim 17, wherein (a) is employed in an amount of from about 0.1 to about 20% by weight, based on the weight of the composition.

23) The method of claim 17, wherein (b) is a hydrophobically modified mica.

24) The method of claim 17, wherein (b) is employed in an amount of from about 0.1 to about 99% by weight, based on the weight of the composition.

25) The method of claim 17, wherein (c) is employed in an amount of from about 5 to about 90% by weight, based on the weight of the composition.
